(19) United States
(12) Patent Application Publication
(10) Pub. No.: US 2023/0101834 A1
(43) Pub. Date: Mar. 30, 2023

(54) PADLOCK PROBE-BASED ROLLING CIRCLE AMPLIFICATION PAIRED WITH NUCLEASE PROTECTION FOR POINT-OF-NEED NUCLEIC ACID DETECTION

(71) Applicant: COLORADO STATE UNIVERSITY RESEARCH FOUNDATION, Fort Collins, CO (US)

(72) Inventors: Brian J. GEISS, Fort Collins, CO (US); Charles S. HENRY, Fort Collins, CO (US); David S. DANDY, Fort Collins, CO (US); Sidhartha JAIN, Fort Collins, CO (US); Devon OSBOURNE, Fort Collins, CO (US)

(21) Appl. No.: 17/906,310

(22) PCT Filed: Mar. 14, 2021
(86) PCT No.: PCT/US2021/022260

§ 371 Date: Sep. 14, 2022

Related U.S. Application Data
(60) Provisional application No. 63/039,799, filed on Jun. 16, 2020.
(60) Provisional application No. 62/989,592, filed on Mar. 14, 2020.

(51) Int. Cl. C12Q 1/70; B01L 3/00

(52) U.S. Cl. CPC C12Q 1/701; B01L 3/5023; C12Q 1/6804

(57) ABSTRACT

The invention discloses a method and a system to detect a target nucleic acid sequence in a sample using padlock probe-based rolling circle amplification and nuclease protection. Padlock probe-based rolling circle amplification and nuclease protection may be used in combination with other detection assays to detect target nucleic acid sequences in a sample.

### CROSS REFERENCE TO RELATED APPLICATIONS

This present invention claims the benefit of the U.S. patent application Ser. No. 62/989,592, filed on 14 Mar. 2020 and entitled PADLOCK PROBE-BASED ROLLING CIRCLE AMPLIFICATION PAIRED WITH NUCLEASE PROTECTION FOR POINT-OF-NEED NUCLEIC ACID DETECTION and the U.S. patent application Ser. No. 63/039,799, filed on 16 Jun. 2020 and entitled PADLOCK PROBE-BASED ROLLING CIRCLE AMPLIFICATION PAIRED WITH NUCLEASE PROTECTION FOR POINT-OF-NEED NUCLEIC ACID DETECTION.

### FIELD OF INVENTION

This invention relates to methods for the sequence-specific detection of nucleic acids.

Embodiments of the present invention relates to a method for the detection and quantification of a specific nucleic acid sequence in a sample. More specifically, the invention provides a method for detecting the presence of a specific nucleic acid using padlock probed-based rolling circle amplification (RCA) and nuclease protection in combination with other detection assays. The method of the invention is particularly useful for in vitro diagnostic application.

### BACKGROUND OF THE INVENTION

Ultrasensitive, sequence-specific detection of a target nucleic acid sequence has broad ranging applications in clinical diagnostics, water and food safety, environmental monitoring, biosafety, epidemiology, and more. With the introduction of polymerase chain reaction (PCR) and other DNA amplification techniques, such as recombinase polymerase amplification, template-mediated amplification, helicase-dependent amplification, loop-mediated isothermal amplification and rolling circle amplification, significant progress has been made in the field of molecular diagnostics and nucleic acid biosensors.

Nucleic acid amplification is a pivotal process in biotechnology and molecular biology and has been widely used in research, medicine, agriculture and forensics. PCR was the first nucleic acid amplification method developed and until now has been the method of choice since its invention. PCR is often used as a method for nucleic acid amplification for its well-known methodology, extensively validated standard operating procedure, and availability of reagents and equipment. However, PCR has many limitations, including high cost of equipment, sensitivity to certain classes of contaminants and inhibitors, requirement for optimized primers, requirement of thermal cycling, and frequency of sequencing errors.

These limitations gave birth to alternative methods such as rolling circle amplification (RCA). RCA is a mechanism used in nature for the replication of circular DNA such as plasmids in bacteria. The reaction has been adopted as the basis for a laboratory method for amplifying circular molecules and, as well as having utility in methods of amplifying or producing nucleic acids, has been demonstrated to be useful in a variety of assays which use or generate a circular nucleic acid molecule as a reporter; in such assay methods the circular molecule is amplified (replicated) by RCA and the replicated or amplified circular nucleic acid sequence is detected. As the rolling circle template is endless, the resultant reaction product is a long concatenated single-stranded nucleic acid molecule composed of tandem repeats, or monomers, that are complementary to the padlock probe.

However, the reaction product of RCA requires further modifications to detect and analyze the reaction product. Hence, there is a need for a novel method and system to rapidly detect a target nucleic acid sequence using RCA and analyze the resulting reaction product.

### SUMMARY OF THE INVENTION

The present invention is directed to a method and a system to detect a target nucleic acid sequence in a sample using padlock probe-based rolling circle amplification (RCA) and nuclease protection in combination with other detection assays, as set forth in or otherwise apparent from the description and drawings that follow, and that which is learned by the practice of the subject matter disclosed herein.

In an embodiment of the invention, a method for detecting a target nucleic acid sequence in a sample comprises the steps of providing a sample, padlock probes, other components for rolling circle amplification, nuclease protection probes, and nucleases. The padlock probes hybridize to the target nucleic acid sequence of interest to form a circular probe for RCA. Following hybridization, RCA amplifies the target nucleic acid sequence, where the RCA reaction product contains binding sites for the nuclease protection probes. Following hybridization of the nuclease protection probes, the single-stranded sections of the RCA reaction product are digested using nucleases. The resulting reaction products are the double-stranded nuclease protection probes. The double-stranded nuclease protection probes may be labeled and detected for analysis.

The invention also discloses a system to detect a target nucleic acid sequence in a sample, the system comprising a platform, one or more heaters, a sample loading zone, one or more channels, a padlock probe zone, a ligation zone, an RCA zone, a protection zone, a nuclease digestion zone, and a detection zone.

The foregoing may cover only some of the aspects of the invention. Other and sometimes more particular aspects of the invention will be appreciated by reference to the following description of at least one preferred mode for carrying out the invention in terms of one or more examples. The following mode(s) for carrying out the invention are not a definition of the invention itself but are only example(s) that embody the inventive features of the invention.

### BRIEF DESCRIPTION OF THE DRAWINGS

For a fuller understanding of the invention, reference should be made to the following detailed description, taken in connection with the accompanying drawings, in which:

FIG. 1 is a schematic of the padlock probe-based RCA and nuclease protection assay in accordance with an embodiment of the system and of the method of detecting a target nucleic acid sequence using padlock probe-based rolling circle amplification paired with nuclease protection. The assay involves hybridization of a padlock probe to the target nucleic acid sequence. Upon hybridization the two ends of that padlock probe may be ligated to form a circular probe. The circular probe may undergo rolling circle amplification in the presence of DNA or RNA polymerase and deoxynucleotides. A nuclease protection probe is added to the reaction that binds to the concatenated single-stranded nucleic acid. Nuclease digestion cleaves single-stranded DNA and unhybridized nuclease protection probe. Double-stranded nuclease protection probes are protected from nuclease digestion and detected using various detection methods.

FIG. 2 is a schematic showing a lateral flow assay as a detection method for the double-stranded protection probes in accordance with an embodiment of the system and of the method of detecting a target nucleic acid sequence using padlock probe-based rolling circle amplification paired with nuclease protection.

FIG. 3 is a schematic showing exponential RCA (eRCA) used for the detection of target nucleic acid sequences in accordance with an embodiment of the system and of the method shown in FIG. 2.

FIG. 4 is an image of a gel and lateral flow strips showing padlock probe-based rolling circle amplification and nuclease protection assay feasibility in accordance with an embodiment of the system and of the method shown in FIG. 2.

FIG. 5 is an image of a graph and lateral flow strips showing padlock probe-based rolling circle amplification and nuclease protection assay sensitivity in accordance with an embodiment of the system and of the method shown in FIG. 2.

FIG. 6 is a schematic of an embodiment of the system using padlock probe-based rolling circle amplification and nuclease protection assay.

FIG. 7 is a schematic of an embodiment of the system using padlock probe-based rolling circle amplification nuclease protection assay in combination with a lateral flow assay.

### DETAILED DESCRIPTION OF THE INVENTION

The present invention is a method and system of detecting sequence specific nucleic acids using padlock probe-based rolling circle amplification (RCA) and nuclease protection. The invention provides a simple, cost-effective and ultrasensitive method and system for the detection of nucleic acids.

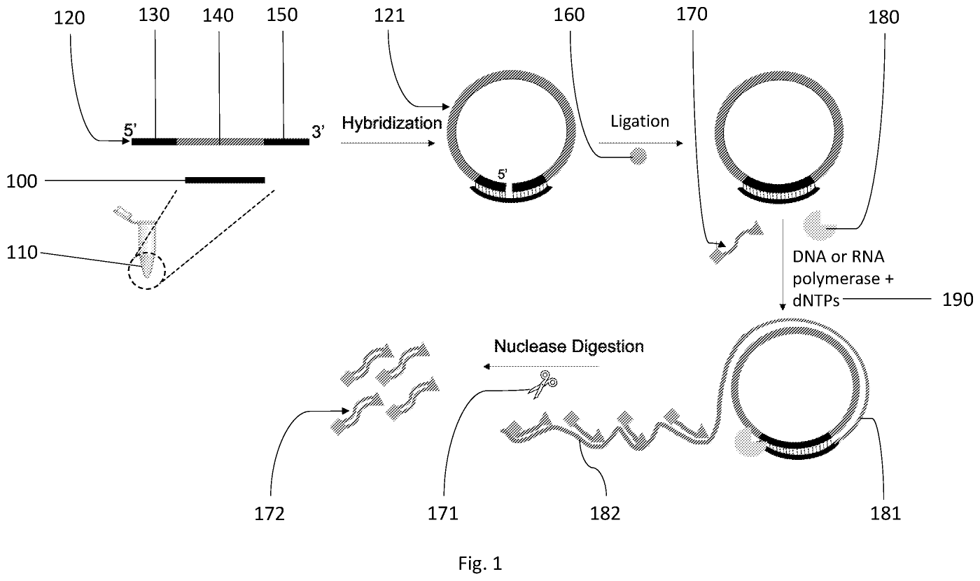

With reference to FIG. 1, a method for the detection of a target nucleic acid sequence 100 of interest in a sample 110 comprises a padlock probe 120 based RCA and nuclease protection. First, the following are provided: a sample 110 to be screened for presence of a target nucleic acid sequence 100; a plurality of padlock probes 120, each padlock probe 120 comprising a 5′ end 130, a 3′ end 150, and a central region 140, wherein the 5′ ends 130 and the 3′ ends 150 of each padlock probe 120 comprise sequences complementary to the target nucleic acid sequence 100 and the central regions 140 comprise a sequence that matches a sequence on a nuclease protection probe 170; a plurality of ligases 160; a plurality of DNA or RNA polymerases 180; a plurality of deoxynucleotides (dNTPs) 190; a plurality of nuclease protection probes 170; and a plurality of nucleases 171.

In an embodiment of the invention, the sample 110 may be any sample that contains nucleic acids or genetic material including, but not limited to, saliva, urine, blood, serum, plasma, and fecal matter, and the target nucleic acid sequence 100 may be DNA or RNA and may be of any origin including, but not limited to, bacterial, viral, parasitic, and human and non-human animals. The sample 110 may be obtained from any multi-cellular organism including, but not limited to, human and non-human mammals, birds, fish, and amphibians. In an embodiment of the invention, the sample 110 is from a subject that is known or suspected of having an infection with a virus or other pathogen, wherein the pathogen has a characteristic nucleic acid sequence serving as a target for detection.

In an embodiment of the invention, padlock probes 120 may be a nucleic acid molecule capable of hybridizing with a target nucleic acid sequence molecule and, when hybridized to the target, is capable of being detected either directly or indirectly. Padlock probes 120 may permit the detection, and, in some examples, quantification, of a target nucleic acid sequence molecule, such as a DNA or RNA.

In an embodiment of the invention, the sample 110 may be exposed to high temperature conditions to facilitate denaturing of nucleic acids. Denaturing of nucleic acids prepares the sample 110 for hybridization to probes.

In an embodiment of the invention, the padlock probes 120 may be oligonucleotides that are short stretches of single-stranded DNA or RNA. The 5′ end 130 and 3′ end 150 of each padlock probe 120 are specific to the target nucleic acid sequence 100. The 5′ end 130 and 3′ end 150 contain sequences that are complementary to the target nucleic acid sequence 100 so that when the 5′ end 130 and 3′ end 150 hybridize to the target nucleic acid sequence 100, the 5′ end 130 and 3′ end 150 are immediately next to each other, forming a circularized probe 121. The circular probe 121 may serve as a template for RCA. Uncircularized padlock probes are unable to serve as a template for rolling circle amplification. After hybridization, the circularized probe 121 on the target nucleic acid sequence 100 is missing a phosphodiester bond between the 5′ end 130 and 3′ end 150, which may be ligated using ligases 160. In an embodiment of the invention, each 5′ end 130 of the padlock probe 120 may be phosphorylated to facilitate ligation.

In an embodiment of the invention, after hybridization, adenosine triphosphate (ATP) and a ligation buffer may be added to the RCA reaction to activate the ligases 160. T4 DNA ligase may be used as the ligase 160.

Following ligation, in an embodiment of the invention, the plurality of DNA or RNA polymerases 180 and the plurality of dNTPs 190 are added to facilitate RCA. An RCA buffer may be added to facilitate RCA. The circular probe 121 acts as a circular template for nucleic acid amplification. The RCA reaction produces a concatenated single-stranded nucleic acid 181 complementary to the sequence on the padlock probes 120. The central region 140 of each padlock probe 120 contains a sequence that matches the nuclease protection probes 170. When the padlock probes 120 are amplified by RCA, the concatenated single-stranded nucleic acid 181 contains a plurality of binding sites 182 that are complementary to the nuclease protection probes 170.

In an embodiment of the invention, Phi29 buffer and bovine serum albumin (BSA) may be added to the RCA reaction and Phi29 DNA polymerase may be used as the DNA or RNA polymerase 180.

As the RCA reaction is ongoing, the plurality of nuclease protection probes 170 is added and the plurality of nuclease protection probes 170 binds to the plurality of binding sites 182 on the concatenated single-stranded nucleic acid 181. The nuclease protection probe 170 may be a single-stranded nucleic acid molecule having a sequence that is complementary to a target DNA or RNA and is capable of hybridizing to the target DNA or RNA. In this embodiment, the plurality of nuclease protection probes 170 is complementary to the plurality of binding sites 182 on the concatenated single-stranded nucleic acid 181.

Following hybridization of the plurality of nuclease protection probes 170 to the plurality of binding sites 182 on the concatenated single-stranded nucleic acid 181, the plurality of nucleases 171 is added to digest single-stranded nucleic acids. After hybridization, the concatenated single-stranded nucleic acid 181 contains doubled-stranded sections where the nuclease protection probes 170 hybridized and single-stranded sections where the nuclease protection probes 170 did not hybridize. The nuclease protection probe 170 protects the double-stranded sections from cleavage by the nuclease 171.

Nucleases are enzymes that cleave a phosphodiester bond. In an embodiment of the invention, the nucleases may be P1 nucleases, which are zinc-dependent single-strand specific nucleases that hydrolyze phosphodiester bonds in RNA and single-stranded DNA with no base specificity. P1 nuclease buffer may also be added to facilitate the activity of P1 nucleases. The resulting reaction product after nuclease digestion is a plurality of double-stranded nuclease protection probes 172.

Following nuclease digestion, the presence, concentration, or quantity of the plurality of double-stranded nuclease protection probes 172 may be detected using various techniques including, but not limited to, gel electrophoresis, genetic sequencing, electrochemical detection, and lateral flow assay. The mode of detection may depend on how the nuclease protection probe 170 is labelled.

In other embodiments of the invention, the nuclease protection probe 170 may further comprise one or more detectable labels or a moiety to facilitate detection. Detection may occur through immobilization of the nuclease protection probe 170 to a solid substrate.

In other embodiments of the invention, the label for the nuclease protection probe 170 may be any moiety that provides for signal detection and may vary widely depending on the particular nature of the assay. Label moieties of interest include both directly and indirectly detectable labels. Suitable labels for use include, but not limited to, any moiety that is indirectly or directly detectable by spectroscopic, photochemical, biochemical, immunochemical, electrical, optical, chemical, or other means. Suitable labels include, but not limited to, antigenic labels including, but not limited to, digoxigenin, fluorescein, dinitrophenol, biotin for staining with labeled streptavidin conjugate, fluorescent dyes including, but not limited to fluorescein, Texas red, rhodamine, fluorophore labels including, but not limited to, an ALEXA FLUOR® label, radiolabels including, but not limited to, 3H, 1251, 35S, 14C, and 32P, enzymes including, but not limited to, peroxidase, alkaline phosphatase, galactosidase, and others commonly used in an ELISA, fluorescent proteins including, but not limited to, green fluorescent protein, red fluorescent protein, yellow fluorescent protein, synthetic polymers chelating a metal, colorimetric labels. An antigenic label may also be incorporated into the nucleic acid on any nucleotide.

In embodiments of the invention, fluorescent labels may be detected using a photodetector to detect emitted light, enzymatic labels are typically detected by providing the enzyme with a substrate and detecting the reaction product produced by the action of the enzyme on the substrate, colorimetric labels may be detected by simply visualizing the colored label, and antigenic labels may be detected by providing an antibody (or a binding fragment thereof) that specifically binds to the antigenic label. An antibody that specifically binds to an antigenic label may be directly or indirectly detectable. For example, the antibody may be conjugated to a label moiety that provides the signal or the antibody may be conjugated to an enzyme that produces a detectable signal when provided with an appropriate substrate.

Following detection, data obtained from detection may be analyzed to determine the presence, concentration, or quantity of target nucleic acid sequence 100 in the sample 110. In an embodiment of the invention, data will be compared to output data of a known concentration or quantity of nucleic acid to determine the concentration or quantity of the sample 110. Data quantification methods will vary based on detection mode. For colorimetric reactions, detection may be through visual analysis or imaging. For fluorescence or chemiluminescence modes, detection is achieved by quantifying light emission. For electrochemical detection, quantification is achieved by electrons transferred through amperometry, voltammetry, or potentiometry.

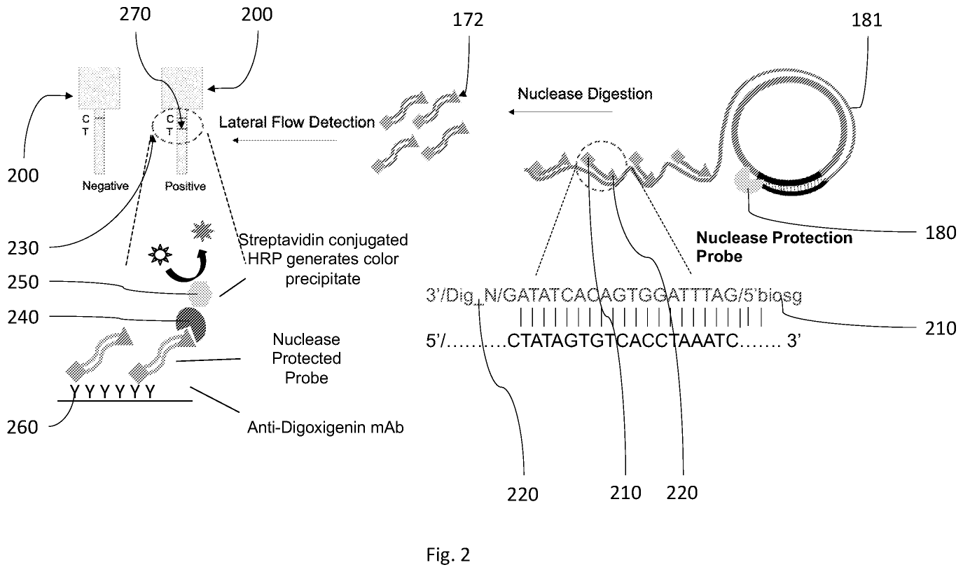

With reference to FIG. 2, in an embodiment of the invention, a lateral flow assay is used to detect the presence of the double-stranded nuclease protection probe 172. In such embodiment, the nuclease protection probes 170 may be labelled with a label for detection. The nuclease protection probe 170 may comprise a 5′ biotin label 210 and a 3′ digoxigenin 220 label. A lateral flow assay strip 200 comprises a detection region 230, a reporter enzyme 240 and a colorimetric substrate 250. The detection region 230 is treated with a monoclonal antibody 260 that binds to a label on the nuclease protection probe 170. In an embodiment of the invention, the monoclonal antibody 260 may be an anti-digoxigenin antibody that binds to the 3′ digoxigenin label 220. The 5′ biotin 210 may be used to show a visible signal of the presence of the double-stranded nuclease protection probe 172. The reporter enzyme 240 may be a streptavidin-conjugated reporter enzyme. Addition of the streptavidin-conjugated reporter enzyme 240 and the colorimetric substrate 250 to the reaction mix is used to generate a colorimetric signal 270 following binding of the streptavidin-conjugated reporter enzyme 240 to the 5′ biotin 210 label. The appearance of the colorimetric signal 270 at the detection region 230 on the lateral flow assay strip 200 indicates the presence of the target nucleic acid sequence 100 in the sample 110. The intensity of the signal may be quantified to estimate the concentration or quantity of the target nucleic acid sequence 100 in the sample 110. RCA and nuclease protection may be performed at a constant temperature, thereby eliminating the need for a thermocycler for strict temperature control. The isothermal condition is to be at a temperature that is optimized for the hybridization of the padlock probes 120 and nuclease protection probes 170 to the target nucleic acid sequence 100 and the concatenated single-stranded nucleic acid 181, respectively.

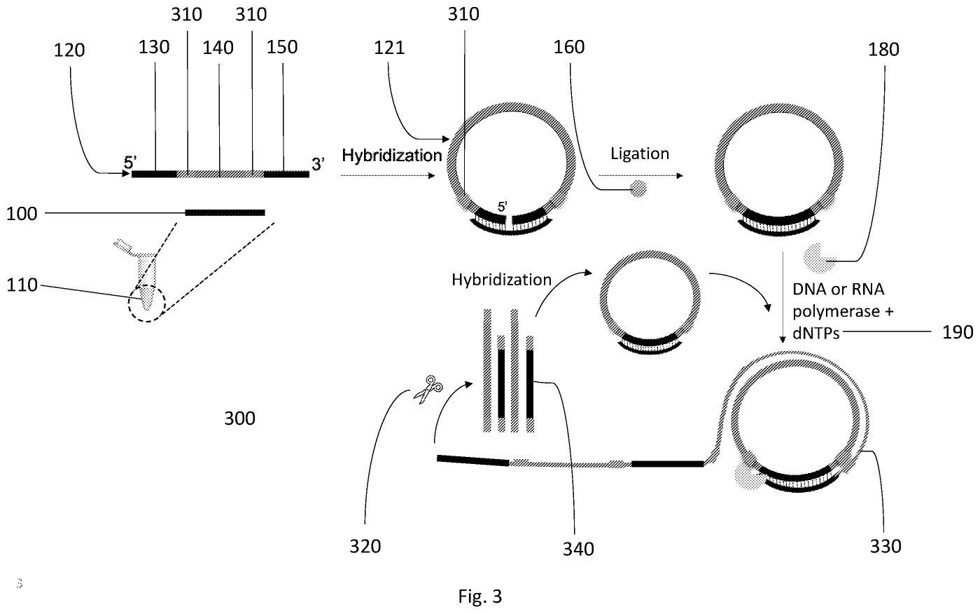

With reference to FIG. 3, in other embodiments of the invention, exponential RCA (eRCA) 300 is performed in lieu of RCA. For eRCA, the padlock probe 120 comprises the 5′ end 130, the 3′ end 150, the central region 140, and two strand-specific nuclease recognition sites 310. The strand-specific nuclease recognition sites 310 are located between the 5′ end 130 and the central region 140 and between the 3′ end 150 and the central region 140. The strand-specific nuclease recognition sites 310 may comprise a specific sequence that a sequence-specific nuclease 320 may recognize and nick the nucleic acid at that precise site. Similar to RCA, the 5′ end 130 and 3′ end 150 of each padlock probe 120 are specific to the target nucleic acid sequence 100. The 5′ end 130 and 3′ end 150 contain sequences that are complementary to the target nucleic acid sequence 100 so that when the 5′ end 130 and 3′ end 150 hybridize to the target nucleic acid sequence 100, the 5′ end 130 and 3′ end 150 are immediately next to each other, forming the circularized probe 121. The resulting circularized probe 121 on the target nucleic acid sequence 100 is missing a phosphodiester bond between the 5′ end 130 and 3′ end 150, which may be ligated using ligases 160. Each 5′ end 130 of the padlock probe 120 may be phosphorylated to facilitate ligation.

After hybridization and ligation, the plurality of DNA or RNA polymerases 180 and the plurality of dNTPs 190 are added to facilitate RCA. The RCA reaction produces a concatenated single-stranded nucleic acid 330 complementary to the sequence on the padlock probes 120 with strand-specific nuclease recognition sites 310. After one cycle of RCA, the sequence specific nuclease 320 is added to nick the concatenated single-stranded nucleic acid 330 at the strand-specific nuclease recognition sites 310. The result is shorter sections of single-stranded nucleic acid 340. Since these shorter sections of single-stranded nucleic acid 340 contain the target nucleic acid sequence 100 sequence, padlock probes 120 may hybridize onto the shorter sections of single-stranded nucleic acid 340 and begin RCA. This cycle repeats itself and there is an increased amplification of the target nucleic acid sequence 100.

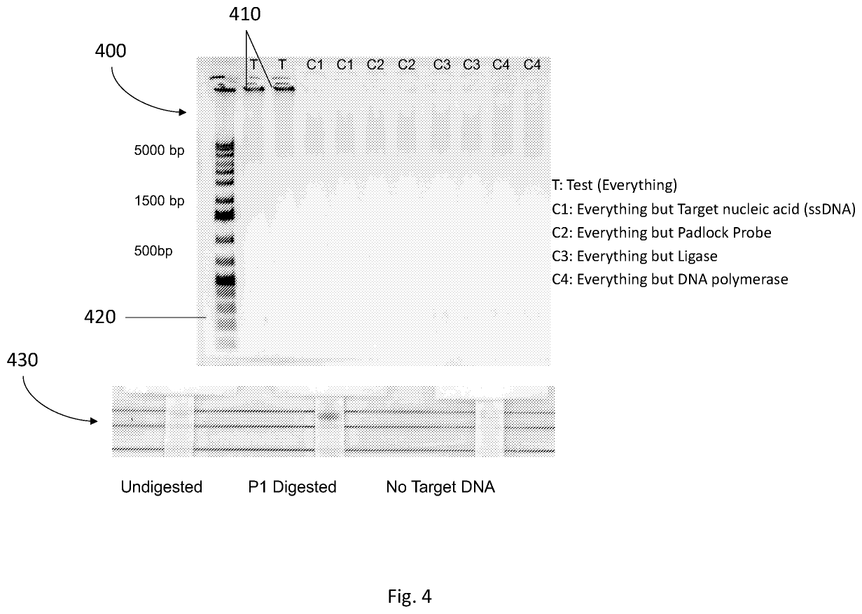

With reference to FIG. 4, in an embodiment of the invention, the product of RCA may be separated using gel electrophoresis, which separates nucleic acid by size, for detection. The shorter nucleic acids will travel down the agarose gel 420 much quicker than longer nucleic acids. Since the concatenated single-stranded nucleic acid 181 is long as a result of RCA, the concatenated single-stranded nucleic acid 181 is shown as a signal 410 close to the top of the agarose gel 420. The concatenated single-stranded nucleic acid 181 is only produced by the RCA reaction if the target nucleic acid sequence 100, the padlock probe 120, the ligase 160, and the DNA or RNA polymerase 180 are all added. If any one of the components are missing, RCA does not occur and the concatenated single-stranded nucleic acid 181 is not produced. As a result, there is no signal when any one of the components are not added to the reaction.

The gel electrophoresis results show that only the test condition (T) produces signal as a large molecular weight DNA band appears in the well of the agarose gel 420. Controls (C1-C4), where one of the RCA reaction components was not added, do not produce any signal. On the lateral flow strips 430, the sample 110 with no target nucleic acid sequence does not produce signal (No Target DNA), while nuclease 171 digested positive sample containing target nucleic acid sequence 100 produces a blue test line. The nuclease 171 undigested positive sample did not produce a test line indicating that without nuclease 171 digestion, the nuclease protection probe 170 remains trapped on the concatenated single-stranded nucleic acid 181 and does not flow in the lateral flow assay strip 200. Nuclease 171 digestion cleaves single-stranded nucleic acid, freeing up the double-stranded nuclease protection probe 172 for detection in the lateral flow assay strip 200.

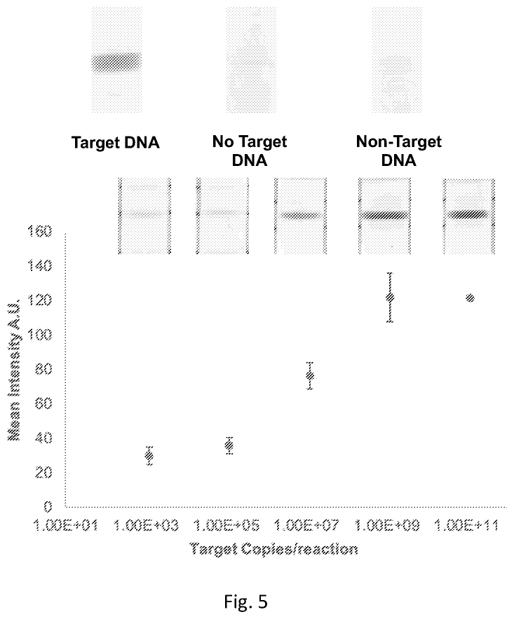

With reference to FIG. 5, in an embodiment of the invention, the concatenated single-stranded nucleic acid 181 is only produced when you add the target nucleic acid sequence 100 to the RCA reaction and the concentration or quantity of the concatenated single-stranded nucleic acid 181 produced is dependent on the initial concentration of target nucleic acid sequence 100. As the concentration of target nucleic acid sequence 100 increases, the concentration or quantity of the concatenated single-stranded nucleic acid 181 produced also increases. In such embodiment of the invention, the method may detect target nucleic acid sequence 100 in concentrations as low as 2 zmol (2×10⁻²¹ mol).

In an embodiment of the invention shown in FIG. 5, no target refers to a reaction condition where there is no nucleic acid target in the reaction. In such condition, there is just water, padlock probes 120, enzymes, and buffer. Non target refers to a non-specific target that should not hybridize with the padlock probe 120. In this condition, there is the padlock probe 120, non-specific target oligo, enzymes, and buffer. With reference to FIG. 5, the lateral flow assay strip shows a signal only when the target nucleic acid sequence 100 is in the sample 110. In the presence of non-specific nucleic sequences (non target), there is no signal on the lateral flow assay strip.

In other embodiments of the invention, the target nucleic acid sequence 100 may be any polynucleotide nucleic acid molecule including, but not limited to, DNA molecules, RNA molecules, and modified nucleic acids present in a sample 110 or to be screened for the presence of said target in a sample 110. The target nucleic acid sequence 100 may also be a coding RNA (mRNA) or a non-coding RNA (tRNA, rRNA, microRNA (miRNA), mature miRNA, immature miRNA). The target nucleic acid sequence 100 may also be a splice variant of an RNA molecule or an unspliced RNA including, but not limited to, pre-mRNA, mRNA, a partially spliced RNA, or a fully spliced RNA.

In other embodiments of the invention, the target nucleic acid sequence 100, in various embodiments, may be one that is found in a biological organism including, but not limited to, a microorganism or infectious agent, or any naturally occurring, bioengineered or synthesized component thereof. In other embodiments of the invention, the target nucleic acid sequence 100 may be or contain a portion of a gene, a regulatory sequence, genomic DNA, cDNA, RNA including mRNA, rRNA, microRNA, small interfering RNA (siRNA), small nuclear RNA (snRNA), doubled-stranded RNA (ds RNA) or any combination thereof. The target nucleic acid sequences of interest may be variably expressed, wherein the methods of the invention allow profiling and comparison of the expression levels of nucleic acids, including without limitation RNA transcripts. A target nucleic acid sequence may also be a denatured genomic molecule or other variants including, but not limited to, viral nucleic acids, bacterial nucleic acids and plasmids.

In other embodiments of the invention, the target nucleic acid sequence 100 is a sequence from a virus including, but not limited to, HIV, HCV, SARS-CoV-2 and dengue. In such embodiments, the method may detect whether a subject is infected by such virus.

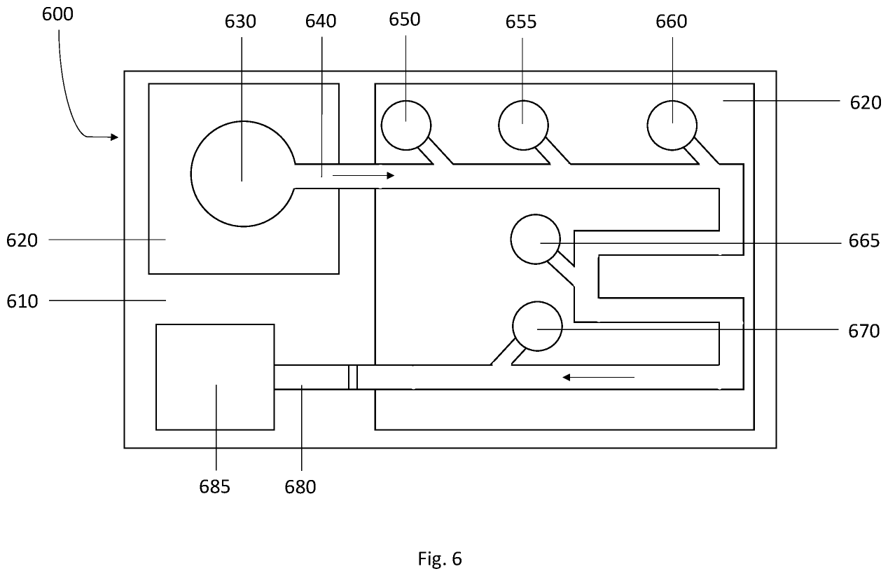

With reference to FIG. 6, a system 600 for the detection of a target nucleic acid sequence 100 in a sample 110 comprises: a platform 610; one or more heaters 620; a sample loading zone 630; a channel 640; a padlock probe zone 650 comprising a plurality of padlock probes 120, each padlock probe 120 comprising the 5′ end 130, the 3′ end 150, and the central region 140, wherein the 5′ ends 130 and the 3′ ends 150 of the padlock probes 120 comprise sequences complementary to the target nucleic acid sequence 100 and the central regions 140 comprise a sequence that matches a sequence on the nuclease protection probes 170; a ligation zone 655 comprising a plurality of ligases 160, a ligation buffer, and a plurality of adenosine triphosphates (ATPs); an RCA zone 660 comprising, a plurality of DNA or RNA polymerases 180, an RCA buffer, a plurality of dNTPs 190, and a plurality of BSA; a protection zone 665 comprising a plurality of nuclease protection probes 170; a nuclease digestion zone 670 comprising, a plurality of nucleases 171, and a nuclease buffer; a detection zone 680; and an absorbent pad 685.

In an embodiment of the invention, the sample 110 is added to the sample loading zone 630 on the system 600. The heater 620 may be set at a temperature high enough to denature the sample 110 and prepare the sample 110 for detection. The sample 110 may be in a sample buffer solution to facilitate flowing through the system via capillary action. Capillary action induces flow of the sample 110 through the channel 640 (as shown by an arrow) into the padlock probe zone 650.

The padlock probe zone 650 comprises a plurality of padlock probes 120. The padlock probes 120 may be dehydrated and the sample 110 in the buffer solution may hydrate the dehydrated padlock probes 120. The plurality of padlock probes 120 hybridize to the plurality of target nucleic acid sequence 100 on the sample 110 to form the circular probe 121. Following hybridization, the circular probe 121 and the sample 110 flow through the channel 640 to the ligation zone 655. At the ligation zone 655, the plurality of ligases 160 ligate the 5′ end 130 and the 3′ end 150 of the circular probe 121. The ligases 160, the ligation buffer, and the ATPs may be dehydrated, and require hydration by the sample buffer solution.

Following ligation, the circular probe 121 and the sample 110 flow to the RCA zone 660 where the plurality of DNA or RNA polymerases 180 facilitate RCA. The DNA or RNA polymerases 180, the RCA buffer, the dNTPs 190, and the plurality of BSA may be dehydrated and require hydration by the sample buffer solution. The components of the RCA zone 660 facilitate RCA and produce the concatenated single-stranded nucleic acids 181.

Following RCA, the concatenated single-stranded nucleic acids 181 flow through the channel 640 to the protection zone 665. In the protection zone, the plurality of nuclease protection probes 170 hybridize onto the binding sites 182 on the concatenated single-stranded nucleic acids 181. The nuclease protection probes 170 may be dehydrated and required hydration by the sample buffer solution.

Following hybridization, the hybridized concatenated single-stranded nucleic acids 181 and nuclease protection probes 170 flow through the channel 640 to the nuclease digestion zone 680. At the nuclease detection zone 680, the plurality of nucleases 171 digests the single-stranded sections on the concatenated single-stranded nucleic acids 181. The reaction product is the plurality of double-stranded nuclease protection probes 172. The nucleases and nuclease buffer may be dehydrated and require hydration by the sample buffer solution.

In an embodiment of the invention, the padlock probe zone 650, the ligation zone 655, the RCA zone 660, the protection zone 665, and the digestion zone 670 all function in an isothermal condition. The heater 620 may be set at a temperature optimized for hybridization, ligation, RCA and digestion reactions.

In an embodiment of the invention, the flow of the sample may be controlled at each zone using passive or active valves that prevent the sample 110 and the sample buffer from flowing to the next zone. Flow is controlled by capillary forces. This allows for different incubation periods between the zones.

Following digestion, the double-stranded nuclease protection probes 172 flow to the detection zone 680. The absorbent pad 685 facilitates capillary action by absorbing the sample buffer solution, which allows the double-stranded nuclease protection probes 172 to flow through the detection zone 680. The presence and concentration or quantity of the plurality of double-stranded nuclease protection probes 172 may be detected using various techniques including, but not limited to, gel electrophoresis, genetic sequencing, and lateral flow assay. The mode of detection may depend on how the nuclease protection probe 170 is labelled.

In other embodiments of the invention, the nuclease protection probe 170 may further comprise one or more detectable labels or a moiety to facilitate detection. Detection may occur through immobilization of the nuclease protection probe 170 to a solid substrate.

In other embodiments of the invention, the label for the nuclease protection probe 170 is any moiety that provides for signal detection and may vary widely depending on the particular nature of the assay. Label moieties of interest include both directly and indirectly detectable labels. Suitable labels for use include, but not limited to, any moiety that is indirectly or directly detectable by spectroscopic, photochemical, biochemical, immunochemical, electrical, optical, chemical, or other means. Suitable labels include, but not limited to, antigenic labels including, but not limited to, digoxigenin, fluorescein, dinitrophenol, biotin for staining with labeled streptavidin conjugate, fluorescent dyes including, but not limited to fluorescein, Texas red, rhodamine, fluorophore labels including, but not limited to, ALEXA FLUOR® label, radiolabels including, but not limited to, 3H, 1251, 35S, 14C, and 32P, enzymes including, but not limited to, peroxidase, alkaline phosphatase, galactosidase, and others commonly used in an ELISA, fluorescent proteins including, but not limited to, green fluorescent protein, red fluorescent protein, yellow fluorescent protein, synthetic polymers chelating a metal, colorimetric labels. An antigenic label may also be incorporated into the nucleic acid on any nucleotide.

In embodiments of the invention, fluorescent labels may be detected using a photodetector to detect emitted light, enzymatic labels are typically detected by providing the enzyme with a substrate and detecting the reaction product produced by the action of the enzyme on the substrate, colorimetric labels may be detected by simply visualizing the colored label, and antigenic labels may be detected by providing an antibody (or a binding fragment thereof) that specifically binds to the antigenic label. An antibody that specifically binds to an antigenic label may be directly or indirectly detectable. For example, the antibody may be conjugated to a label moiety that provides the signal or the antibody may be conjugated to an enzyme that produces a detectable when provided with an appropriate substrate.

Following detection, in an embodiment of the invention, data obtained from detection may be analyzed to determine the concentration or quantity of target nucleic acid sequence 100 in the sample 110. In an embodiment of the invention, data will be compared to output data of a known concentration or quantity of nucleic acid to determine the concentration or quantity of the sample 110. Data may also be compared to real-time reverse transcriptase PCR or digital droplet PCR.

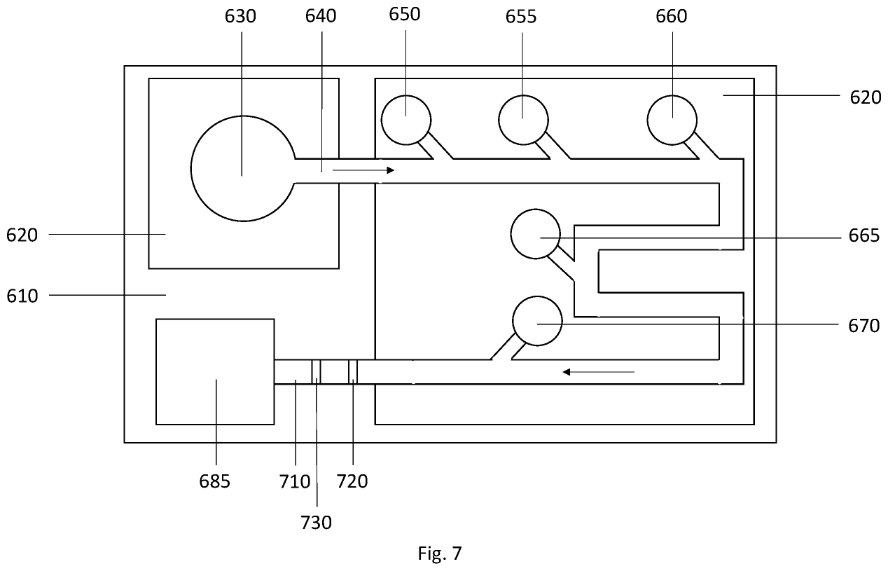

With reference to FIG. 7, in an embodiment of the invention, a lateral flow assay is used to detect the presence of the double-stranded nuclease protection probe 172. In such embodiment, the nuclease protection probes 170 may be labelled with a label for detection. The nuclease protection probe 170 may comprise a 5′ biotin label 210 and a 3′ digoxigenin 220 label.

A lateral flow assay detection zone 710 may comprise a colorimetric reagent zone 720 comprising the reporter enzyme 240 and the colorimetric substrate 250 and a test line 730 comprising the monoclonal antibody 260 that binds to a label on the nuclease protection probe 170. In an embodiment of the invention, the monoclonal antibody 260 may be an anti-digoxigenin antibody that binds to the 3′ digoxigenin label 220. The 5′ biotin 210 may be used to show a visible signal of the presence of the double-stranded nuclease protection probe 172 on the test line 730. The reporter enzyme 240 may be a streptavidin-conjugated reporter enzyme. Addition of the streptavidin-conjugated reporter enzyme 240 and the colorimetric substrate 250 to the colorimetric reagent zone 720 is used to generate a colorimetric signal 270 at the test line 730 following binding of the streptavidin-conjugated reporter enzyme 240 to the 5′ biotin 210 label. The appearance of the colorimetric signal 270 at the test line 730 on the lateral flow assay detection zone 710 indicates the presence of the target nucleic acid sequence 100 in the sample 110. The intensity of the signal may be quantified to estimate the concentration or quantity of the target nucleic acid sequence 100 in the sample 110.

In the foregoing description, exemplary modes for carrying out the invention in terms of examples have been described. However, the scope of the claims should not be limited by those examples but should be given the broadest interpretation consistent with the description as a whole. The specification and drawings are, accordingly, to be regarded in an illustrative rather than a restrictive sense.

1. A method for detection a target nucleic acid sequence in a sample, the method comprising the steps of:
providing:
a sample to be screened for presence of a target nucleic acid sequence of interest;
a plurality of padlock probes, each padlock probe comprising a 5′ end, a 3′ end, and a central region, wherein the 5′ ends and the 3′ ends of each padlock probe comprise sequences complementary to the target nucleic acid sequence and the central regions of each padlock probe comprise a sequence that matches a sequence on a nuclease protection probe;
a plurality of ligases;
a plurality of DNA or RNA
polymerases; a plurality of
deoxynucleotides (dNTPs);
a plurality of nuclease protection probes;
and a plurality of nucleases;
hybridizing the plurality of padlock probes to the plurality of target nucleic acid sequence of interest, wherein the 5′ end and the 3′ end of each padlock probe are brought immediately next to each other upon hybridization with the target nucleic acid sequence to form a circular probe;
adding the ligase for facilitating ligation of the 5′end and 3′ end of each padlock probe of the plurality of padlock to form a circular template;
adding the plurality of DNA or RNA polymerases and the plurality of dNTPs for facilitating rolling circle amplification (RCA), wherein the RCA reaction produces a concatenated single-stranded nucleic acid complementary to the sequence on the padlock probes, and wherein the concatenated single-stranded nucleic acid comprises a plurality of binding sites complementary to the sequence on the nuclease protection probes;
adding the plurality of nuclease protection probes for binding to the binding sites on the concatenated single-stranded nucleic acid and forms sections of double-stranded nuclease protection probes;
adding the plurality of nucleases to digest sections of the concatenated single-stranded nucleic;
digesting the single-stranded nucleic acid using the plurality of nucleases to obtain doubled-stranded nuclease protection probes as a reaction product;
detecting the presence, concentration, or quantity of the reaction product; and
analyzing data from detecting the presence, concentration, or quantity of the reaction product.

2. The method of claim 1 wherein the sample is from saliva, urine, blood, serum, plasma, mucus, or fecal matter of the subject.

3. The method of claim 1, wherein each padlock probe comprises a 5′-phosphate modification.

4. The method of claim 1, wherein each nuclease protection probe comprises a label for detection.

5. The method of claim 4 wherein the label for detection may be fluorescent, colorimetric, radioactive, enzymatic, or antigenic.

6. The method of claim 1, wherein detecting the presence, concentration, or quantity of the reaction product further comprises using a lateral flow assay.

7. The method of claim 1, wherein each of the plurality of nuclease protection probe further comprises 5′ biotin.

8. The method of claim 7 further comprising adding a streptavidin-conjugated reporter enzyme and a colorimetric substrate to the captured digoxigenin labeled doubled-stranded nuclease protection probes to visibly show the presence of the target nucleic acid sequence in the sample.

9. The method of claim 1, wherein each of the plurality of nuclease protection probe further comprises a 3′ digoxigenin.

10. The method of claim 9 further comprising detecting the presence of double-stranded nuclease protection probes with an anti-digoxigenin antibody using the lateral flow assay to capture the digoxigenin labeled doubled-stranded nuclease protection probes.

11. The method of claim 1, further comprising measuring the intensity of the signal from an image of the colorimetric signal to estimate quantities of the target nucleic acid sequence in the sample.

12. The method of claim 1, wherein the target nucleic acid sequence is a sequence from a virus selected from the group consisting of HIV, HCV, SARS-CoV-2 and dengue.

13. The method of claim 1, wherein the padlock probe further comprises strand-specific nuclease recognition sites.

14. The method of claim 13 wherein exponential rolling circle amplification (eRCA) is performed in the presence of a DNA or DNA polymerase and dNTPs and multiple concatenated single-stranded nucleic acids are produced.

15. A system for the detection of a target nucleic acid sequence in a sample, the system comprising:
a platform;
one or more heaters;
a sample loading zone;
a channel;
a padlock probe zone comprising,
a plurality of padlock probes, each padlock probe comprising a 5′ end, a 3′ end, and a central region, wherein the 5′ ends and the 3′ ends of the padlock probes comprise sequences complementary to the target nucleic acid sequence and the central regions comprise a sequence that matches a sequence on the nuclease protection probes;
a ligation zone comprising,
a plurality of ligases;
a ligation buffer; and
a plurality of adenosine triphosphate (ATPs);
an RCA zone comprising,
a plurality of DNA or RNA polymerases;
an RCA buffer;
a plurality of dNTPs; and
a plurality of bovine serum albumin;
a protection zone comprising,
a plurality of nuclease protection probes;
a nuclease digestion zone comprising,
a plurality of nucleases;
and a nuclease buffer;
a detection zone; and
an absorbent pad.

16. The system of claim 15 wherein each padlock probe comprises a 5′-phosphate modification.

17. The system of claim 15, wherein each nuclease protection probe comprises a label for detection.

18. The system of claim 15, wherein the label for detection may be fluorescent, colorimetric, radioactive, enzymatic, or antigenic.

19. The system of claim 15, further comprising a lateral flow assay strip to detect the presence, concentration, or quantity of the reaction product.

20. The system of claim 15, wherein each of the plurality of nuclease protection probe further comprises 5′ biotin.

21.-25. (canceled)
